(19) United States
(12) Patent Application Publication
(10) Pub. No.: US 2012/0309973 A1
(43) Pub. Date: Dec. 6, 2012

(54) PREPARATION OF 2-(4-BROMOPHENYL)-2-METHYLPROPANOIC ACID

(75) Inventors: Murali Krishna Prasad Divi, Hyderabad (IN); Gundu Rao Padakandla, Hyderabad (IN); Nageswara Rao Bolneni, Hyderabad (IN); Krishnaji Rao Mutyala, Hyderabad (IN)

(73) Assignee: DIVI'S LABORATORIES LIMITED, Hyderabad (IN)

(21) Appl. No.: 13/219,971

(22) Filed: Aug. 29, 2011

(30) Foreign Application Priority Data
Jun. 6, 2011 (IN) 1908/CHE/2011

(51) Int. Cl. C07D 211/22; C07C 51/363

(52) U.S. Cl. 546/239; 562/496

(57) ABSTRACT

Selective bromination of 2-methyl-2-phenylpropanoic acid in aqueous medium is described to obtain pure 2-(4-bromophenyl)-2-methylpropanoic acid, which is a useful key intermediate in the process of manufacturing pure fexofenadine.

### CROSS-REFERENCE TO RELATED APPLICATION

This application claims priority from India Application 1908/CHE/2011, filed Jun. 6, 2011, entitled Preparation of 2-(4-Bromophenyl)-2-Methylpropanoic Acid, which application is assigned to the same assignee as this application and whose disclosure is incorporated by reference herein.

### FIELD OF INVENTION

Present invention relates to a process for preparation of pure 2-(4-bromophenyl)-2-methylpropanoic acid, substantially free from its isomers, which is required to prepare pure fexofenadine free from its undesirable isomers.

### BACKGROUND OF THE INVENTION

Fexofenadine is (±)-p-[1-hydroxy-4-[4-(hydroxydiphenylmethyl)piperidino]butyl]-α-methylhydratropic acid It is known as an active metabolite of antihistamine terfenadine and is marketed as a non-sedating antihistamine. Its hydrochloride salt is a pharmaceutically acceptable salt form. The USP monograph on fexofenadine hydrochloride prescribes limits for related substances and impurities. The isomeric 3-[1-hydroxy-4-[4-(hydroxydiphenylmethyl)-1-piperidinyl]butyl]-α,α-dimethyl benzeneacetic acid hydrochloride is denoted as ‘fexofenadine related compound B’ in the USP but is limited under ‘any other unknown impurity’ to less than 0.1%. On the other hand the Ph.Eur. limits this impurity specifically to not more than 0.1%. The Indian Pharmacopoeia (Ind.P.) also specifically limits this impurity to less than 0.2%. Both the Ph.Eur. and Ind.P. prescribe a separate test for determining this impurity as distinct from the test for ‘Related substances’. The Pharmacopoeias also limit impurity A (which has the keto function in place of the secondary alcohol function in the side chain of fexofenadine) and the ‘decarboxylated’ impurity. The specified isomeric impurity B arises from the route of synthesis ordinarily followed by manufacturers as described in literature (J. Org. Chem. 1994, 59, 2620-2622). The exact source of this impurity is the key intermediate 2-(4-bromophenyl)-2-methylpropanoic acid, which may contain significant quantities of the isomeric 2-(3-bromophenyl)-2-methyl propanoic acid. This isomeric impurity in the key intermediate undergoes all the subsequent reactions in the route of synthesis and emerges as the impurity B in the fexofenadine (J. Org. Chem., as cited above). The impurity A and ‘decarboxylated’ impurity are relatively easily removed during normal purification steps. However the impurity B, owing to its very close resemblance to the API with similar properties like polarity and solubility, is not so easily removed. Often more than three purification steps are needed to reduce this impurity to the compendia) limits thus reducing yields of the API substantially and increasing cost of production. Hence it makes sense to limit this impurity at the source key intermediate. Highly pure fexofenadine can be obtained by using pure 2-(4-bromophenyl)-2-methylpropanoic acid (formula 1) as the key intermediate substantially free from the meta isomer (formula-2) and ortho isomer (formula-3).

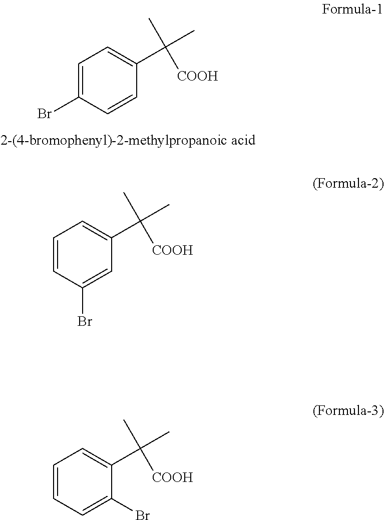

Thus, there is a need for developing an industrial process to produce pure 2-(4-bromophenyl)-2-methylpropanoic acid, which can be used as precursor to make pure fexofenadine.

### DESCRIPTION OF PRIOR ART

The process for preparing methyl 2-(4-bromophenyl)-2-methylpropanoate is disclosed in J. Org. Chem. 1994, 59, 2620-2622. Herein, methyl 2-(4-bromophenyl)-2-methyl propanoate is prepared by esterification of 4-bromophenylacetic acid followed by methylation of the benzylic carbon employing 2.4 equivalents of methyl iodide in presence of sodium hydride in tetrahydrofuran medium. However, the process of selective bromination either from phenyl acetic acid or from 2-methyl-2-phenylpropanoic acid is not described. Our own efforts did not result in industrial application of this process due to following drawbacks.
1) Preparation of 4-bromophenylacetic acid requires multistep synthesis.
2) Trimethyl chlorosilane and methyl iodide reagents are expensive to use on commercial scale.
3) Sodium hydride is highly flammable material and industrially hazardous reagent.
4) Tetrahydrofuran used as solvent is also expensive to use on commercial scale.
5) The reaction is highly exothermic.

The process for preparing 2-(4-bromophenyl)-2-methylpropanoic acid of formula-1 is also disclosed in Journal of the American Chemical Society, 1957, vol. 79, p. 3432. As described therein a mixture of 2-methyl-2-phenylpropanoic acid, bromine and iron wire are refluxed in carbon tetrachloride. After workup, product was isolated after four crystallizations. We investigated this prior art process to prepare desired compound in pure form. The results are indicated in Table-1. We could notice following disadvantages:
1) The bromination reaction is non-selective, simultaneously giving rise to meta and ortho isomers of formula-2 and formula-3 respectively.
2) Bromination reaction is incomplete as indicated by the presence of unreacted starting material.
3) Isolation of pure product is not achieved due to presence of unreacted starting material and impurities.
4) Complete elimination of unreacted starting material and impurities is not achieved even after four recrystallizations.

TABLE 1

| | | Yield | | | | | | | | | |
|---|---|---|---|---|---|---|---|---|---|---|---|
| | | of | G.C composition of crude | | | | | | | | |
| | Batch | crude | product | | | | Yield after | G.C composition of product | | | |
| | scale | product | (% area) | | | | four | After four crystallizations | | | |
| S. | in | in | | | Impurity | | crystallizations | | | Impurity | |
| No | grams | grams | SM | Product | meta | ortho | in grams | SM | Product | meta | ortho |
| 1 | 26.6 | 30.2 | 25.14 | 62.4 | 7.70 | 1.42 | 15.1 | 11.11 | 84.94 | 2.14 | Nil |
| 2 | 26.6 | 30.1 | 35.81 | 47.26 | 5.71 | 1.10 | 10.3 | 15.78 | 65.32 | 2.10 | Nil |
| 3 | 26.6 | 30.0 | 29.73 | 56.51 | 7.59 | 1.68 | 14.6 | 12.98 | 80.23 | 2.18 | Nil |
| 4 | 26.6 | 29.9 | 25.37 | 61.74 | 7.75 | 1.75 | 15.2 | 10.78 | 86.15 | 2.99 | Nil |

SM = Starting material: (2-methyl-2-phenylpropanoic acid);
Product = 2-(4-bromophenyl)-2-methylpropanoic acid;
Impurity: meta = 2-(3-bromophenyl)-2-methyl propanoic acid;
Impurity: ortho = 2-(2-bromophenyl)-2-methylpropanoic acid

Thus prior art processes suffer from following disadvantages and are not suited well for industrial application:
1) Non-selective bromination leading to formation of undesirable meta and ortho isomers (formula-2 and 3 respectively) in the key intermediate. Efforts to eliminate impurities result in low yield of pure product.
2) Reaction is incomplete requiring several recrystallizations to obtain pure product in low yield.
3) Carbon tetrachloride is class-I solvent and toxicologically objectionable for industrial application.

For these reasons a need was felt to find a more efficient way of synthesis. Our efforts resulted in the novel method for selective bromination of 2-methyl-2-phenylpropanoic acid to obtain 2-(4-bromophenyl)-2-methylpropanoic acid, substantially free from its isomers. Unexpectedly it was found that bromination of 2-methyl-2-phenylpropanoic acid could be achieved in aqueous medium in acidic, neutral or alkaline conditions, avoiding undesirable halogenated hydrocarbon solvents with excellent selectivity.

### SUMMARY OF THE INVENTION

The present invention reveals a process for the preparation of 2-(4-bromophenyl)-2-methylpropanoic acid, which comprises of reacting 2-methyl-2-phenylpropanoic acid with bromine in water medium to obtain substantially pure 2-(4-bromophenyl)-2-methylpropanoic acid, a useful intermediate in the process of making pure fexofenadine.

### DETAILED DESCRIPTION OF THE INVENTION

As used herein, 2-methyl-2-phenylpropanoic acid, a substrate for bromination, can be easily prepared from benzene, methacrylic acid and aluminium chloride as described by Prijs, B. in Helvetica Chimica Acta, vol. 35, (1952), p 780 (scheme-1)

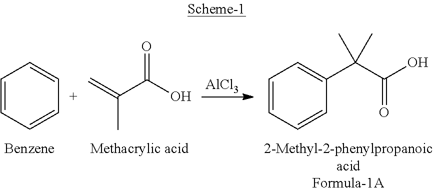

2-(4-Bromophenyl)-2-methylpropanoic acid was prepared by bromination of 2-methyl-2-phenylpropanoic acid in aqueous medium as shown in scheme-2.

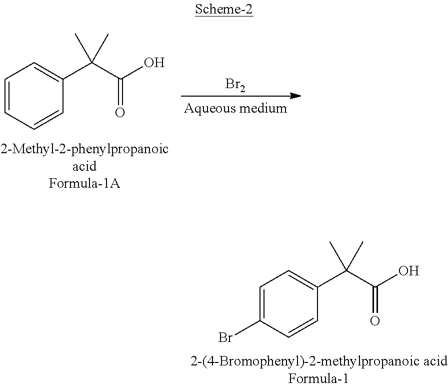

Bromination reaction can be performed in acidic, alkaline or neutral conditions. In aqueous medium, we were pleasantly surprised with the selective bromination to yield 2-(4-bromophenyl)-2-methylpropanoic acid predominantly.

The preparation of 2-(4-bromophenyl)-2-methylpropanoic acid comprises of
reacting 2-methyl-2-phenylpropanoic acid (substrate) with bromine in water medium,
extracting heterogeneous solution with an organic solvent and
recovering the product.

In one embodiment of the invention, the reaction is performed in a heterogeneous condition, wherein solid matter is dispersed in reaction medium under acidic condition. Preferably one to two equivalents of bromine to one equivalent of substrate are adequate for completion of the reaction. Large excess of bromine is not required. At less than one equivalent of bromine input bromination was incomplete. The separation of non-brominated starting material (2-methyl-2-phenylpropanoic acid) from 2-(4-bromophenyl)-2-methylpropanoic acid product is cumbersome by simple crystallization due to similar solubility properties. The product mixture containing starting material can be subjected to esterification, preferably methyl ester, followed by separation by means of distillation under reduced pressure. We observed no significant losses during recovery process as ester form, although it involves additional steps of esterification and separation by distillation.

The solvent for extraction process is selected from a category of water immiscible solvent wherein 2-(4-bromophenyl)-2-methylpropanoic acid is sufficiently soluble. The preferred solvents for extraction are ethers, hydrocarbons, esters, ketones, halo hydrocarbons or alcohols, most preferably dichloromethane.

The combined extracts are preferably dried and concentrated under reduced pressure to recover product as well as solvent. The practically dry residue is preferably suspended in an organic solvent in which the product is practically insoluble, such as hexanes or heptanes and filtered to recover the product. The obtained product showed about 95% content of 2-(4-bromophenyl)-2-methylpropanoic acid and about 5% content of 2-(3-bromophenyl)-2-methylpropanoic acid.

In another embodiment of present invention, selective bromination of 2-methyl-2-phenylpropanoic acid performed in water medium under alkaline condition, in which the mass is in a homogeneous condition. The product is recovered by acidifying with an acid either by filtration or by extraction with a suitable immiscible solvent. The obtained product has about 98% content of 2-(4-bromophenyl)-2-methylpropanoic acid and about 2% of 2-(3-bromophenyl)-2-methylpropanoic acid.

In another embodiment of present invention, selective bromination of 2-methyl-2-phenylpropanoic acid performed in water medium at about pH 7 (neutral condition), wherein neutralization is achieved using alkali solution. The product is recovered by acidification followed by filtration or extraction with a suitable solvent. The obtained product has about 99% content of 2-(4-bromophenyl)-2-methylpropanoic acid and about 1% of 2-(3-bromophenyl)-2-methylpropanoic acid.

The bromination process was monitored by gas chromatographic analysis either directly as acid form or samples can be converted to ester by general method of esterification using methanol and acid catalyst before analysis.

### EXAMPLES

The following examples are for illustration purpose only and do not limit the invention in any way. The reagents and solvents mentioned in examples maybe replaced by other equivalent reagents and solvents known to those skilled in the art.

### Example 1

2-Methyl-2-phenylpropanoic acid (25 g, 0.1524 moles) and water (300 ml) were charged to 500 ml three-necked round bottomed flask at ambient temperature (25° C. to 30° C.). To the resulting suspension, 43.8 g of bromine was added drop wise. Reaction mixture was stirred at 75-80° C. until complete consumption of 2-methyl-2-phenylpropanoic acid as determined by gas chromatographic analysis. The reaction mixture containing precipitated product was cooled to ambient temperature and extracted with dichloromethane (3×75 ml). The extracts were combined, dried with anhydrous sodium sulphate and evaporated to dryness. Resulting solid product was suspended in hexanes (50 ml) and filtered to recover crude product (37 g, quantitative yield), GC purity 94.4% of 2-(4-bromophenyl)-2-methylpropanoic acid and 5.5% of 2-(3-bromophenyl)-2-methylpropanoic acid. Crude product was recrystallized from aqueous methanol to give 29.0 g (78% yield) of G.C purity 99.2% 2-(4-bromophenyl)-2-methylpropanoic acid and 0.79% of 2-(3-bromophenyl)-2-methylpropanoic acid.

### Example 2

2-Methyl-2-phenylpropanoic acid (5 g, 0.0304 moles) and sodium bicarbonate solution in water (23.06 g in 200 ml of water) were charged to 500 ml three-necked round bottomed flask at ambient temperature (25° C. to 30° C.). To the resulting solution, 8.7 g of bromine was added drop wise. Reaction mixture was stirred until complete consumption of 2-methyl-2-phenylpropanoic acid by gas chromatographic analysis. Reaction mixture was acidified with 5N hydrochloric acid to pH 1 to 2. Aqueous solution was extracted with dichloromethane (3×50 ml). The extracts were combined, dried with anhydrous sodium sulphate and evaporated to dryness. Resulting solid product (7.4 g, quantitative yield) was suspended in hexanes (50 ml) and filtered to recover the product (5.5 g, 74.3% yield), GC purity 98.8% of 2-(4-bromophenyl)-2-methylpropanoic acid and 1.18% of 2-(3-bromophenyl)-2-methylpropanoic acid.

### Example 3

2-Methyl-2-phenylpropanoic acid (5 g, 0.0304 moles) and water (50 ml) were taken in to three-necked round bottomed flask at ambient temperature (25° C. to 30° C.). To the resulting mixture, sodium carbonate solution (20% in water) was added drop wise until pH shows about 7. To the resulting solution 8.7 g of bromine was added drop wise while maintaining pH of reaction solution at about 7 by addition of sodium carbonate solution. Reaction mixture was stirred until complete consumption of 2-methyl-2-phenylpropanoic acid as determined by gas chromatographic analysis. The neutral reaction solution was acidified with 5N hydrochloric acid to pH 1 to 2. Aqueous solution was extracted with dichloromethane (3×50 ml). All organic layers were combined, dried with anhydrous sodium sulphate and evaporated to dryness. Resulting solid product was suspended in hexanes (50 ml) and filtered to recover the product, 6.0 g, 81% yield, GC purity 98.5% of 2-(4-bromophenyl)-2-methylpropanoic acid and 1.25% of 2-(3-bromophenyl)-2-methylpropanoic acid.

### Example 4

### Step-1: Preparation of 2-methyl-2-phenylpropanoic acid

This was prepared essentially by the method of Prijs, B (see Scheme-1 above) using benzene (5500 L) and methacrylic acid (550 Kg) in presence of aluminium chloride (2200 Kg). The resulting product of G.C purity 98.0%, was used as such in the next step.

### Step-2: Preparation of 2-(4-bromophenyl)-2-methylpropanoic acid

The wet material (on dry basis) from step-1 (275 Kg, assay 98%) was charged into a reactor containing 6875 L of water and 660 Kg of sodium bicarbonate. Bromine (330 Kg) was fed slowly into the reactor during 3 hours at 25-35° C. and maintained for 10 hours at 25-35° C. Toluene (275 L) was charged into reactor and stirred for 15 minutes. The aqueous phase was discharged into another reactor and pH adjusted to about 5 with dilute hydrochloric acid (880 L of water and 440 L of hydrochloric acid) at 0-10° C. The resultant material was filtered and washed with water. The obtained wet product was treated with heptanes (700 L) to recover 190 Kg (46.6% yield) of 2-(4-bromophenyl)-2-methylpropanoic acid, GC purity: 99.28% of 2-(4-bromophenyl)-2-methylpropanoic acid and 0.72% of 2-(3-bromophenyl)-2-methylpropanoic acid,

### Step-3: Preparation of methyl 2-(4-bromophenyl)-2-methylpropionate

For operational convenience the product from two or three batches of Step-2 are combined and used in this step. The wet material (on dry basis) from step-2 (575 Kg, purity of about 98.2%) was charged into a reactor containing 1150 L of toluene. The mass was maintained for about 15 minutes or until a clear solution was obtained. The toluene layer was washed with sodium chloride solution (20% in water) and separated. To the toluene layer, methanol (300 L) was charged followed by sulphuric acid addition at 25-35° C. The reaction mass temperature was gradually raised to 63-67° C. and stirred for 16 hours or until complete consumption of 2-(4-bromophenyl)-2-methylpropanoic acid as indicated by GC analysis. The mass was gradually cooled to 25-35° C. and then washed with water (3500 L), sodium carbonate solution (2% in water) and sodium chloride (10% solution in water) successively. Organic phase was subjected to distillation under reduced pressure to obtain methyl 2-(4-bromophenyl)-2-methylpropionate (480 Kg, 79% yield, GC purity: 99.2%).

Step-4 Preparation of fexofenadine from methyl 2-(4-bromophenyl)-2-methylpropionate:

A mixture of 3-butyn-1-ol (30.7 grams), tetrakis (triphenyl phosphine) palladium (0) (1.5 grams) and copper iodide (6 grams) was charged to 1 L flask containing methyl 2-(4-bromophenyl)-2-methylpropionate (step-3 material, 75 grams, purity of about 98.2%) and potassium carbonate (120 grams) in toluene (500 ml). Reaction mass temperature gradually raised to reflux temperature and stirred for 24 hours. The resultant dark solution was filtered. The clear filtrate (toluene layer) was washed with saturated ammonium chloride solution (400 ml), 20% sodium chloride solution (400 ml), dried using anhydrous sodium sulphate and concentrated under reduced pressure to obtain constant weight oily material. This oily material (70 grams) dissolved in methanol (250 ml) and added to a mixture of mercuric oxide (5 grams) and 5% sulphuric acid solution in water. Reaction solution was stirred for 2 hours at 25-35° C. The reaction solution diluted with water (500 ml) and basified to pH: 7-8 using ammonium hydroxide solution and extracted with ethylacetate (2×250 ml). Ethylacetate layer was washed with water (3×350 ml), dried with sodium sulphate, filtered and concentrated. Resultant brown colored thick oily material (68 grams) treated with phosphorus oxychloride (23 ml) in dichloromethane (300 ml) at 25-35° C. under stirring for 24 hours. Reaction was quenched in to chilled water (500 ml). Dichloromethane layer was separated, dried with anhydrous sodium sulphate and concentrated to constant weight. The resultant oily material (48 grams) was refluxed with the mixture of azacyclonol (35 gram), potassium carbonate (94 grams) and potassium iodide (1.5 gram) in toluene (250 ml) for 36 hours. Reaction was quenched with chilled water (500 ml) and toluene layer separated. Aqueous layer was extracted with toluene (2×500 ml). Combined toluene layer was dried and concentrated. Resultant crude material (78.4 grams) was dissolved in methanol (200 ml) at 25-35° C. To this solution, sodium borohydrate (9 grams) added lot wise during 30 minutes (Note: addition of sodium borohydride causes exothermic reaction). Reaction mixture stirred for 16 hours, quenched by adding 2% acetic acid in water (20 ml) and stirred for 30 minutes. The resulted solid material filtered and dried at 60° C. The resultant solid material (31.36 grams of fexofenadine methyl ester) was dissolved in methanol (150 ml). To this solution 2N sodium hydroxide solution (150 ml) was added and heated to 70-75° C. for 16 hours. Reaction solution was clarified by filtration through hyflow bed and the clear filtrate was neutralized with 1N hydrochloric acid. Resulting gummy solid was stirred with methanol for 16 hours. Resulting white crystals of fexofenadine was filtered to obtain 23.5 grams of fexofenadine invariably having a purity of 99.9% and ‘impurity-B’ (isomeric impurity [3-[1-hydroxy-4-[4-(hydroxydiphenylmethyl)-1-piperidinyl]butyl]-α,α-dimethyl benzeneacetic acid below detection levels.

We claim:

1) A process for the preparation of 2-(4-bromophenyl)-2-methylpropanoic acid of formula-1 with a purity of greater than about 98% by gas chromatographic analysis and less than about 2% of the isomeric 2-(3-bromophenyl)-2-methylpropanoic acid 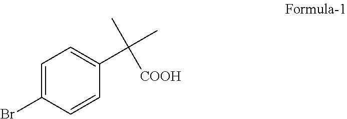
The method comprising bromination of 2-methyl-2-phenylpropanoic acid of formula IA 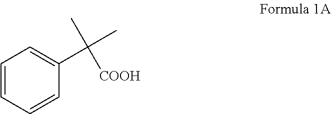
with bromine in an aqueous medium.

2) The process as in claim 1 wherein the aqueous medium is maintained in an acidic or neutral or alkaline condition.

3) A process of preparing fexofenadine with a purity of greater than 99.9% and with no detectable level of impurity B by using the product from the process of claim 1.

4) A process of preparing fexofenadine with a purity of greater than 99.9% and with no detectable level of impurity B by using the product from the process of claim 2.
